(19) United States
(12) Patent Application Publication
(10) Pub. No.: US 2009/0272180 A1
(43) Pub. Date: Nov. 5, 2009

(54) CONTINUOUS TESTING DEVICE AND CONTINUOUS TESTING SYSTEM

(75) Inventors: Yuh-Shyong Yang, Hsinchu City (TW); Ming-Yu Lin, Hsinchu City (TW); Kun-Hsi Tsai, Taichung City (TW); William Wang, Taoyuan County (TW); Long Hsu, Hsinchu City (TW); Cheng-Hsien Liu, Hsinchu City (TW); Chung-Cheng Chou, Taoyuan County (TW)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignees: Yuh-Shyong Yang, Hsinchu City (TW); Long Hsu, Hsinchu City (TW); Cheng-Hsien Liu, Hsinchu City (TW); RAYDIUM SEMICONDUCTOR CORPORATION, Hsinchu (TW)

(21) Appl. No.: 12/272,872

(22) Filed: Nov. 18, 2008

(30) Foreign Application Priority Data
Apr. 30, 2008 (TW) 97115988

(51) Int. Cl. G01N 33/49

(52) U.S. Cl. 73/61.72

(57) ABSTRACT

A continuous testing device for testing the concentration of a target object in a fluid is provided. The continuous testing device includes a first chip, a signal source and a second chip. The first chip includes a separating unit and a reacting unit. The separating unit separates the target object from a non-target object in the fluid. The reacting unit enables the fluid having separated out the non-target object to react with a reagent. The signal source provides a signal passing through the fluid having reacted with the reagent. The second chip disposed at one side of the first chip includes a signal transducing element and a processing unit. The signal transducing element receives the signal passing through the fluid and outputs an electronic signal corresponding to the input signal. The processing unit acquires the concentration of the target object according to the electronic signal.

This application claims the benefit of Taiwan Application Serial No. 097115988, filed Apr. 30, 2008, the subject matter of which is incorporated herein by reference.

### BACKGROUND OF THE INVENTION

1. Field of the Invention

The invention relates in general to a testing device and a testing system, and more particularly to a continuous testing device and a testing system.

2. Description of the Related Art

Many blood tests such as blood sugar concentration, blood cell count and troponin concentration are done by taking blood from the testee. For example, when blood sugar concentration is tested by an individual, the blood sample is tested by a personal blood sugar meter using photo-electro or electro-chemical technology. When the blood sample is tested in a medical center, the blood cells and the blood serum are separated by a centrifuge or a large-scale biochemical analysis instrument first before testing.

Currently, the relevant testing devices for blood sugar and blood serum require independent blood sampling before the blood sample is transferred to the testing center for analysis. Next, the testing personnel will take corresponding actions such as insulin injection according to the results of the testing. Such manual testing is time consuming and cannot provide instant treatment to the patient. Furthermore, the sample may easily be polluted by external objects during the transferring process. Besides, if the sample may cause biochemical pollution, the testing personnel are susceptible to infection. The testing devices which are currently available in the market and using blood cells separation technology such as centrifuge separation technology or capillary separation technology have some disadvantages that severely affect the testing results. For example, the blood cells may easily break and result in hemolysis or may be separated incompletely. Besides, for the patients of many diseases who need to be tested regularly over a long period of the time, conventional manual testing method which requires the patients to be acupunctured repeatedly not only cause inconvenience and decrease infection risk to the patients but also waste medical resources.

### SUMMARY OF THE INVENTION

The invention is directed to a continuous testing device and a continuous testing system. By integrating a separating unit and a reacting unit into the same chip, the fluid sequentially passes through the separating unit and the reacting unit in a continuous testing process. The target object and the non-target object can be separated directly on the chip and the fluid can directly react with the reagent on the chip, so that the concentration of the target object can be instantly tested and acquired. Thus, the concentration of the target object can be continuously monitored over a long period of time and corresponding procedures can be performed according to the change in the concentration of the target object.

According to a first aspect of the present invention, a continuous testing device for testing the concentration of a target object in a fluid is provided. The continuous testing device includes a first chip, a signal source and a second chip. The first chip includes a separating unit and a reacting unit. The separating unit separates the target object from a non-target object in the fluid. The reacting unit enables the fluid having separated out the non-target object to react with a reagent. The signal source provides a signal passing through the fluid having reacted with the reagent. The second chip disposed at one side of the first chip includes a signal transducing element and a processing unit. The signal transducing element receives the signal passing through the fluid and outputs an electronic signal corresponding to the input signal. The processing unit acquires the concentration of the target object according to the electronic signal.

According to a second aspect of the present invention, a continuous testing system for testing the concentration of a target object in a fluid is provided. The continuous testing system includes a continuous testing device and a medicating unit. The continuous testing device includes a first chip, a signal source and a second chip. The first chip includes a separating unit and a reacting unit. The separating unit separates the target object from a non-target object in the fluid. The reacting unit enables the fluid having separated out the non-target object to react with a reagent. The signal source provides a signal passing through the fluid having reacted with the reagent. The second chip disposed at one side of the first chip includes a signal transducing element and a processing unit. The signal transducing element receives the signal passing through the fluid and outputs an electronic signal corresponding to the input signal. The processing unit acquires the concentration of the target object according to the electronic signal. The medicating unit is coupled to the processing unit and used for adjusting a medicating concentration or a medicating frequency according to the concentration of the target object.

The invention will become apparent from the following detailed description of the preferred but non-limiting embodiments. The following description is made with reference to the accompanying drawings.

### BRIEF DESCRIPTION OF THE DRAWINGS

FIG. 1 shows a perspective of a continuous testing system according to a first embodiment of the invention;

FIG. 2 shows a cross-sectional view along the cross-sectional line A-A′ of FIG. 1; and

FIG. 3 shows a perspective of a continuous testing system according to a second embodiment of the invention

### DETAILED DESCRIPTION OF THE INVENTION

The continuous testing system according to a preferred embodiment of the invention integrate a separating unit and a reacting unit on a first chip and integrates a signal transducing element and a processing unit on a second chip, wherein the separating unit is used for separating the target object from a non-target object in the fluid and the reacting unit is used for enabling the fluid to react with the reagent. After the fluid has separated out the non-target object by the separating unit, the fluid can react with the reagent directly on the first chip and further receive the signal passing through the fluid by the signal transducing element to acquire the concentration of the target object. Thus, the information of the concentration of the target object can be acquired continuously, and corresponding procedures can be processed at the same time according to the concentration of the target object. A first embodiment and a second embodiment of the invention are disclosed below for elaborating the purpose of the invention not for limiting the scope of protection of the invention. Furthermore, secondary elements are omitted in the drawings of the embodiments to highlight the technical features of the invention.

### First Embodiment

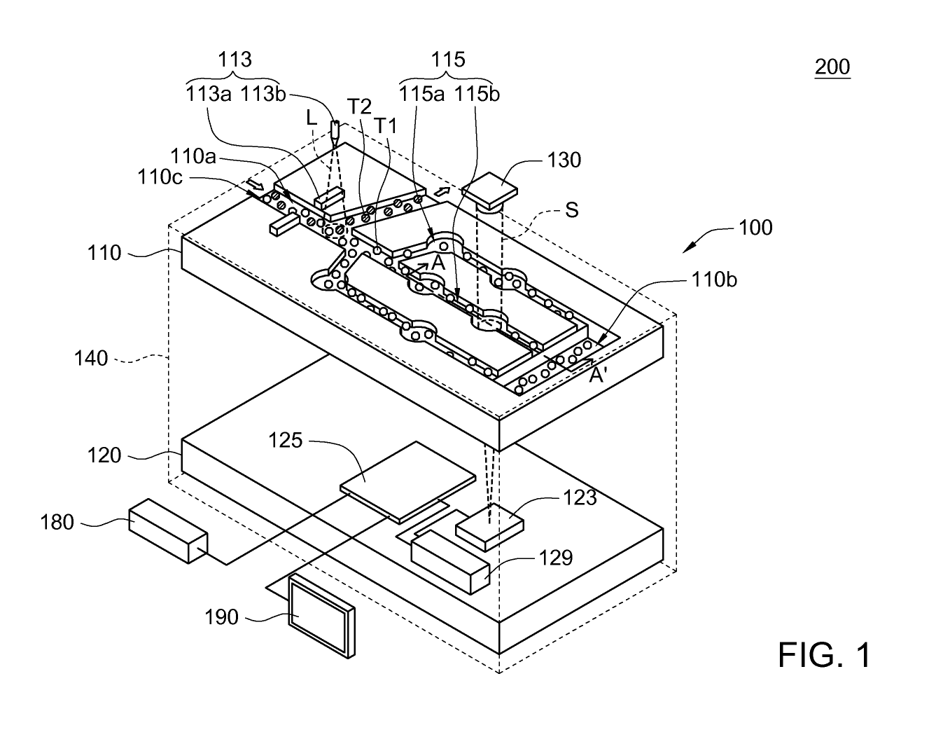

Referring to FIG. 1, a perspective of a continuous testing system according to a first embodiment of the invention is shown. The continuous testing system 200 mainly includes a continuous testing device 100 used for testing the concentration of a target object T1 in a fluid. The continuous testing device 100 includes a first chip 110, a signal source 130 and a second chip 120. The first chip 110 includes a separating unit 113 and a reacting unit 115. The separating unit 113 is for separating the target object T1 from a non-target object in the fluid T2. The reacting unit 115 is for enabling the lo fluid having separated out non-target object T2 to react with a reagent. The signal source 130 provides a signal S passing through the fluid having reacted with the reagent. The second chip 120 disposed at one side of the first chip 110 includes a signal transducing element 123 and a processing unit 125. The signal transducing element 123 receives the signal S passing through the fluid and outputs an electronic signal according to the received signal. The processing unit 125 receives the electronic signal and acquires the concentration of the target object T1 according to the electronic signal T1 in the fluid. Besides, the continuous testing system 200 further includes a medicating unit 180 coupled to the processing unit 125 and adjusts a medicating concentration or a medicating frequency according to the concentration of the target object T1. The continuous testing system 200 uses the separating unit 113 to separate a non-target object T2 from the fluid and increases the precision of testing the concentration of the target object T1. Next, the fluid and the reagent directly react with each other in the reacting unit 115 and the concentration of the target object T1 is tested at the same time. Thus, the testing time is reduced and the medicating unit 180 is capable of making corresponding adjustment according to the concentration of the target object T1.

Furthermore, the first chip 110 has a main fluidic channel 110a used for connecting the separating unit 113 and the reacting unit 115 to transfer the fluid. The main fluidic channel 110a forms a fluid entrance 110c at one side of the first chip 110, wherein the fluid having the target object T1 and the non-target object T2 enter the continuous testing device 100 via the fluid entrance 110c. Examples of the separating unit 113 includes an electrode group 113a disposed at two sides of the main fluidic channel 110a to generate a dielectrophoretic force (DEP force) in the fluid for separating the target object T1 from the non-target object T2 in the fluid. Furthermore, the separating unit 113 of the present embodiment of the invention includes an optical tweezers 113b in addition to the electrode group 113a disclosed above, wherein the optical tweezers 113b provides a focused light L (such as a laser beam) to the fluid. When the focused light L is projected to the fluid, a force is acted on the target object T1 and non-target object T2 in the fluid due to the transfer of the photon momentum of the focused light L. The optical tweezers 113b separates the target object T1 and non-target object T2 by changing the movement direction of the target object T1 and the non-target object T2 according to the wavelength, intensity distribution and focusing angle of the focused light L and the shapes, refractive index and absorptivity of the target object T1 and the non-target object T2. Anyone who is skilled in the technology of the invention will understand the operations of the optical tweezers 113b, and the operations of the optical tweezers 113b are not repeated here. As indicated in FIG. 1, on the part of the continuous testing system 200 of an embodiment of the invention, the separating unit 113 includes the electrode group 113a and the optical tweezers 113b so as to effectively separate the target object T1 from the non-target object T2 in the fluid. However, in different implementations, the separating unit 113 can dispose the electrode group 113a at two sides of the main fluidic channel 110a or use the optical tweezers 113a as a separating mechanism for separating the target object T1 and the non-target object T2. On the other hand, the separated non-target object T2 can be transferred to leave the first chip 110 and stored or wasted according to actual needs.

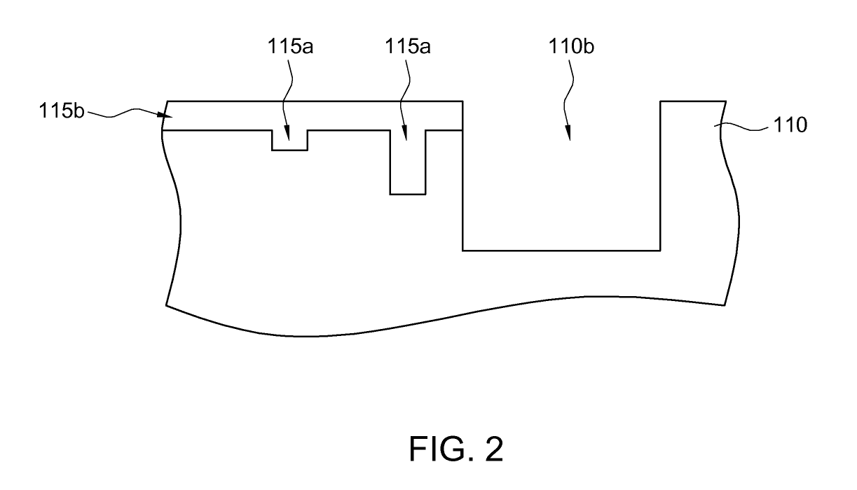

On the other hand, the reacting unit 115 of the present embodiment of the invention includes at least one reaction chambers 115a and many micro-fluidic channels 115b, but is exemplified by including many reaction chambers 115a. The micro-fluidic channels 115b connect the main fluidic channel 110a and the reaction chambers 115a, and the fluid passing through the separating unit 113 enters the reaction chambers 115a via the micro-fluidic channels 115b. The reaction chambers 115a accommodate the fluid and the reagent so that the fluid and the reagent react with each other. After the fluid has reacted with the reagent, the concentration of the target object T1 in the fluid is tested. In the present embodiment of the invention, the reagent is transferred to the reaction chambers 115a via a reagent transmission unit (not illustrated in the diagram) for example. The first chip 110 can be a semiconductor chip, and the reaction chambers 115a and the micro-fluidic channels 115b can be formed on the first chip 110 in a photolithography process. Furthermore, the first chip 110 includes a waste liquid slot 110b connected to the micro-fluidic channels 115b and disposed at the rear of the reacting unit 115 to accommodate the fluid and the reagent which have been reacted and tested. The waste liquid slot 110b can be formed concurrently with the reaction chambers 115a and the micro-fluidic channels 115b in the photolithography process. Referring to FIG. 2, a cross-sectional view along the cross-sectional line A-A′ of FIG. 1 is shown. The reaction chambers 115a preferably have sufficient space for a period of time so that the fluid and the reagent can stay in the reaction chambers 115a and fully react with each other. Moreover, the size of the reaction chambers 115a and how the reaction chambers 115a and connected to the micro-fluidic channels 115b are determined according to actual needs and are not further restricted in the present embodiment of the invention. Furthermore, as the fluid entering the reaction chambers 115a has separated out the non-target object T2, the non-target object T2 will not interfere with the concentration test of the target object T1 and the precision of test will be increased.

In the present embodiment of the invention, the signal source 130 is a light-emitting element such as a LED, the signal S passing through the fluid having reacted with the reagent is a light signal, and the signal transducing element 123 is a photo-electro transducer. In practical application, the part of the first chip 110 corresponding to the reaction chambers 11Sa is made from a transparent material. When the light-emitting element emits a light signal towards the reaction chambers 115a, the light signal passes through the fluid and the first chip 110 passing through the reaction chambers 115a and then is projected onto a photo-electro transducer. The photo-electro transducer is used for detecting the intensity or color of the light having been absorbed by the fluid and then outputting the electronic signal to the processing unit 125. The processing unit 125, according to the electronic signal, operates the concentration of the target object T1 in the fluid. In the present embodiment of the invention, the second chip 120 is a semiconductor chip, the signal transducing element 123 and the processing unit 125 are together formed on the second chip 120 in an integrated semiconductor manufacturing process. As the manufacturing process and procedures of the continuous testing device 100 are simplified, the efficiency of the manufacturing process is increased and the cost is reduced.

The continuous testing device 100 further includes a casing 140, wherein the first chip 110, the signal source 130 and the second chip 120 are all disposed inside the casing 140 as indicated in FIG. 1. In the embodiment of the invention, the first chip 110 is replaceable disposed inside the casing 140, such that the continuous testing device 100 can perform different fluid tests by replacing the first chip 110 and avoid the mixture and pollution of different fluids. Furthermore, the continuous testing device 100 further includes a battery 129 coupled to the signal source 130 and the second chip 120 to provides a potential to the signal source 130 and the second chip 120. The battery 129 is disposed inside the casing 140, such that the continuous testing device 100 can function without being connected to an external power.

Besides, the continuous testing system 200 further includes a display unit 190 coupled to the processing unit 125 to display a frame of testing results according to the concentration of the target object T1 so that the user can conveniently acquire instant information of the testing.

The continuous testing system 200 of the first embodiment of the invention is exemplified by the application in the test of blood sugar concentration. The testee's blood is transferred to the first chip 110 of the continuous testing device 100 by a sample transmission unit (such as a syringe). The sample transmission unit is connected to the testee and the fluid entrance 110c. Then, the blood is transferred to the separating unit 113 via the main fluidic channel 110a, and then the blood cells (the non-target object T2) are separated from the blood by the separating unit 113. The blood serum containing blood sugar (the target object T1) is then transferred to the reacting unit 115. In the reacting unit 115, the blood serum is transferred to the reaction chambers 115a via the micro-fluidic channels 115b, and the blood sugar molecules of the blood serum react with the reagent in the reaction chambers 115a. The reaction chambers 115a preferably have sufficient space so that the blood sugar and the reagent can stay in the reaction chambers 115a for a period of time and fully react with each other. Next, the signal source 130 such as an LED provides a light signal passing through the reacted blood serum to examine the blood sugar concentration according to the photo absorption reaction of the blood serum. The signal transducing element 123 receives the light passing through the blood serum and outputs the electronic signal to the processing unit 125 according to the intensity of the light. The processing unit 125 performs comparison and operation according to the electronic signal to acquire blood sugar concentration. The display unit 190 displays a frame of testing results according to the blood sugar concentration acquired by the processing unit 125, so that the testing personnel will understand whether the blood sugar concentration is normal or not. Furthermore, the medicating unit 180 adjusts the concentration of the medicine injected to the testee and the time interval of injection according to the blood sugar concentration acquired by the processing unit 125 so as to adjust the testee's blood sugar concentration. On the other hand, the tested blood serum is then transferred to the waste liquid slot 110b and stored in the continuous testing device 100, hence avoiding the blood serum leaving the continuous testing device 100 and reducing the risk of infection and pollution. Furthermore, when a testee's blood is tested, the testing personnel only need to withdraw the first chip 110 from the casing 140 and place another first chip into the casing 140. Thus, the risks of mutual infection and errors in sample are largely avoided.

The continuous testing system 200 of the first embodiment of the invention tests blood sugar concentration by continuously testing the sample acquired from the testee at a fixed time interval and quantity. Blood sugar concentration can be tested directly without having to be off-line, and the medicating unit 180 can timely adjust the medicating concentration and the medicating frequency. The continuous testing system 200 has the advantages of making the test of blood sugar concentration faster with higher precision and avoiding the used needles polluting the environment or causing blood infection. The continuous testing system 200 of the first embodiment of the invention is exemplified in the testing of blood sugar concentration. However, the technology of the invention embodiment is not limited thereto. The continuous testing system 200 of the present embodiment of the invention can also be used in other chemical, medical, biological testing or any other fluid test requiring continuous testing over a long period of time.

### Second Embodiment

The continuous testing system of the present embodiment of the invention mainly differs with the continuous testing system of the first embodiment of the invention in the design of the first chip, and other similarities are omitted and are not repeated here.

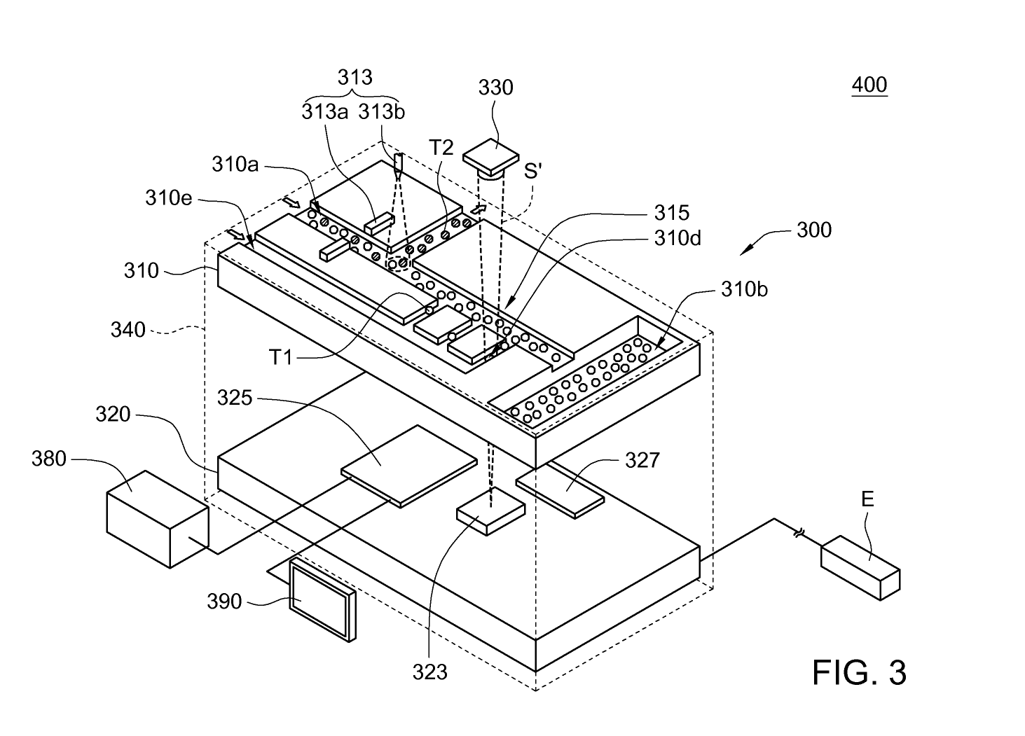

Referring to FIG. 3, a perspective of a continuous testing system according to a second embodiment of the invention is shown. The continuous testing system 400 includes a continuous testing device 300 and a medicating unit 380. The continuous testing device 300 includes a first chip 310, a signal source 330 and a second chip 320. The first chip 310 includes a separating unit 313 and a reacting unit 315. The separating unit 313 used for separating the target object T1 from the non-target object T2 in the fluid includes an electrode group 313a or an optical tweezers 313b, or may also include an electrode group 313a and an optical tweezers 313b. In a preferred embodiment, the reacting unit 315 includes at least one reaction channel 310d, the two ends of the reaction channel 310d respectively receive the fluid and the reagent, and the fluid and the reagent enter the reaction channel 310d due to electrowetting effect and react with each other. The signal source 330 provides a signal S′ passing through the fluid having reacted with the reagent. The signal S′ may be a light signal passing through the fluid and the reagent which are positioned in the reaction channel 310d. The second chip 320 includes a signal transducing element 323, a processing unit 325 and a function generator 327. The function generator 327 provides a wave signal to the reaction channel 310d for enabling the reaction channel 310d to generate an electrowetting effect. Furthermore, the function generator 327 may further provide a wave signal to the electrode group 313a to change the volume and pattern of the DEP force according to the variety and characteristics of the non-target object T2.

Furthermore, the reaction channel 310d is formed on the first chip 310 together with a main fluidic channel 310a and a waste liquid slot 310b in the same photolithography process. The first chip 310 further has a reagent transfer channel 310e, wherein one end of the reagent transfer channel 310e is connected to a reagent slot (not illustrated in the diagram) for transferring the reagent to the first chip 310 and the other end of the reagent transfer channel 310e is connected to the reaction channel 310d. As the hydrophobic or hydrophilic performance on the side wall of the reaction channel 310d is changed by the electrowetting effect, the reaction channel 310d controls the fluid and the reagent in the main fluidic channel 310a to enter the reaction channel 310d and react with each other. On the other hand, the electrowetting effect further enables the fluid and the reagent in the reaction channel 310d to form a focusing liquid drop for focusing the light signal such that the signal transducing element 323 can receive the signal S′ with higher accuracy and the quantity of the fluid and the reagent can be reduced.

Moreover, the continuous testing system 400 of the present embodiment of the invention is connected to an external power E for providing a stable potential to the electrode group 313a, the optical tweezers 313b, the signal source 330 and the second chip 320. The continuous testing device 300 may further include a casing 340, wherein the first chip 310, the signal source 330 and the second chip 320 are disposed inside the casing 340. Furthermore, the continuous testing system 400 may further include a display unit 390 for displaying a frame of testing results.

According to the continuous testing system disclosed in the first and the second embodiment of the invention, the separating unit and the reacting unit are integrated into one single chip for reducing the volume of the testing device. Moreover, as the information of the concentration of the target object can be continuously acquired by way of continuous testing, the testing system can perform corresponding procedures simultaneously and achieve real-time monitoring. Furthermore, the testing process is not off-line, hence preventing the fluid from being exposed and polluted or external objects from entering and polluting the fluid. Also, the signal transducing element, the processing unit and the function generator can be together formed in an integrated semiconductor manufacturing process, further reducing the costs and procedures of the manufacturing process. Besides, the first chip can be directly replaced, hence avoiding the pollution between different fluids and the infection between different testees. Furthermore, the problem arising when the particles of the fluid stuck on the pipe wall obstruct the flow in the channel and affect the testing can be quickly resolved by replacing the first chip. Next, an electrowetting effect can be formed in the reaction channel to generate a focusing liquid drop for increasing the accuracy of testing and reducing the quantity of the fluid and the reagent.

While the invention has been described by way of example and in terms of a preferred embodiment, it is to be understood that the invention is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A continuous testing device for testing the concentration of a target object in a fluid, wherein the continuous testing device comprises:
a first chip, comprising:
a separating unit for separating the target object from a non-target object in the fluid; and
a reacting unit for enabling the fluid having separated out the non-target object to react with a reagent;
a signal source for providing a signal passing through the fluid having reacted with the reagent; and
a second chip disposed at the one side of the first chip, wherein the second chip comprises:
a signal transducing element for receiving the signal passing through the fluid and outputting an electronic signal corresponding to the input signal; and
a processing unit for acquiring the concentration of the target object according to the electronic signal.

2. The continuous testing device according to claim 1, wherein the separating unit comprises:
an electrode group for generating a dielectrophoretic force (DEP force) in the fluid to separate the target object from the non-target object in the fluid, wherein the electrode group prevents the target object and the non-target object from being adhered onto the first chip.

3. The continuous testing device according to claim 1, wherein the separating unit comprises:
an optical tweezers for providing a focused light in the fluid to separate the target object from the non-target object in the fluid.

4. The continuous testing device according to claim 1, wherein the reacting unit comprises:
at least one reaction chamber used for accommodating the fluid and the reagent; and
a plurality of micro-fluidic channels for connecting the at least one reaction chamber.

5. The continuous testing device according to claim 4, wherein the at least one reaction chamber and the micro-fluidic channels are formed on the first chip in a photolithography process.

6. The continuous testing device according to claim 4, wherein the first chip further comprises:
a waste liquid slot connected to the micro-fluidic channels and disposed at the rear of the reacting unit for accommodating the fluid and the reagent which have reacted with each other.

7. The continuous testing device according to claim 1, wherein the reacting unit comprises:
at least one reaction channel whose two ends respectively receive the fluid and the reagent, wherein the at least one reaction channel, due to electrowetting effect, controls the fluid and the reagent to enter the at least one reaction channel to react with each other.

8. The continuous testing device according to claim 7, wherein the at least one reaction channel is formed on the first chip in a photolithography process.

9. The continuous testing device according to claim 7, wherein the fluid and the reagent in the at least one reaction channel further form a focusing liquid drop by the electrowetting effect.

10. The continuous testing device according to claim 7, wherein the second chip further comprises:
a function generator for providing a wave signal to the at least one reaction channel to generate the electrowetting effect.

11. The continuous testing device according to claim 1, further comprising:
a casing, wherein the first chip, the signal source and the second chip are disposed inside the casing;
wherein the first chip is replaceable disposed inside the casing.

12. The continuous testing device according to claim 1, wherein the signal source is a light-emitting element and the signal is a light signal.

13. The continuous testing device according to claim 12, wherein the signal transducing element is a photo-electro transducer.

14. The continuous testing device according to claim 1, further comprising:
a battery coupled to the signal source and the second chip to provide a potential to the signal source and the second chip.

15. The continuous testing device according to claim 1, wherein the first chip has a main fluidic channel for connecting the separating unit and the reacting unit to transfer the fluid.

16. The continuous testing device according to claim 1, wherein the signal transducing element and the processing unit are formed on the second chip in an integrated semiconductor manufacturing process.

17. A continuous testing system for testing the concentration of a target object in a fluid, wherein the continuous testing system comprises:
a continuous testing device, comprising:
a first chip, comprising:
a separating unit for separating the target object from a non-target object in the fluid; and
a reacting unit for enabling the fluid having separated out the non-target object to react with a reagent;
a signal source for providing a signal passing through the fluid having reacted with the reagent; and
a second chip disposed at the one side of the first chip, wherein the second chip comprises:
a signal transducing element for receiving the signal passing through the fluid and outputting an electronic signal corresponding to the input signal; and
a processing unit for acquiring the concentration of the target object according to the electronic signal; and
a medicating unit coupled to the processing unit and used for adjusting a medicating concentration or a medicating frequency according to the concentration of the target object.

18. The continuous testing system according to claim 17, wherein the continuous testing system is connected to an external power to provide at least a potential to the signal source and the second chip.
